(19) United States
(12) Patent Application Publication
(10) Pub. No.: US 2007/0123754 A1
(43) Pub. Date: May 31, 2007

(54) NON-ENCUMBERING, SUBSTANTIALLY CONTINUOUS PATIENT DAILY ACTIVITY DATA MEASUREMENT FOR INDICATION OF PATIENT CONDITION CHANGE FOR ACCESS BY REMOTE CAREGIVER

(76) Inventors: Paul E. Cuddihy, Ballston Lake, NY (US); Jenny M. Weisenberg, Niskayuna, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/164,577

(22) Filed: Nov. 29, 2005

(51) Int. Cl. A61B 5/00; A61B 5/103; A61B 5/117

(52) U.S. Cl. 600/300; 600/595; 128/920

(57) ABSTRACT

An apparatus in one example comprises a user wearable, non-encumbering, movement sensor and an analyzer. The user wearable, non-encumbering, movement sensor substantially continuously measures daily-activity data of a patient. The analyzer makes a determination from the daily-activity data of the patient that a change in condition of the patient has occurred. The analyzer generates an indication of the change in condition of the patient for access by a remote caregiver.

### BACKGROUND OF THE INVENTION

The invention relates generally to health care and more particularly to patient data for patient health care.

A patient who has a health condition and is at home may have both formal and family caregivers who monitor the appearance of the patient. If the patient starts slowing down, sleeping in, not getting dressed, or tiring easily, the patient or caregiver may or may not report the change and get care for the patient. A delay in reporting the change or seeking medical attention may cause harm to the patient. Patient self-reporting can be inconsistent.

Subjectivity by the patient or caregiver may also cause the patient to request medical attention too frequently or infrequently. The requests for attention and visits to medical professionals when not needed consume resources in the health system that could deny timely care to other patients. In addition, the unneeded visits take time and effort of the patient and may expose the patient to additional dangers in making the visit. Waiting too long before receiving medical attention may result in an expensive and more dangerous acute medical event.

Typical measurements from in-home monitoring devices are taken once a day and may miss clear symptoms like worsening sleep or fatigue. The introduction of actual measuring devices into the home can create burdens on the daily routine of the patient. Traditional measurements often involve cumbersome equipment. Being tethered to a substantial measuring device may restrict the patient from movement that may unfortunately worsen the medical condition. The patient may feel awkward in moving and become less motivated to maintain activity. The patient may feel less agile and risk falling. If the patient is disconnected from the device while making movements, then data may be lost or monitoring of the patient compromised. The disconnection could occur by choice of the patient to allow the patient to perform a move unencumbered. The disconnection could occur by accident in movement of the patient and device creating a malfunction in connection and/or measuring.

It would therefore be desirable to promote a convenience and/or continuity in measurement of patient data for communication with a remote caregiver.

### BRIEF DESCRIPTION OF THE INVENTION

In accordance with one aspect of the invention, an apparatus comprises a user-wearable, non-encumbering, movement sensor and an analyzer. The user-wearable, non-encumbering, movement sensor substantially continuously measures daily-activity data of a patient. The analyzer makes a determination from the daily-activity data of the patient that a change in condition of the patient has occurred. The analyzer generates an indication of the change in condition of the patient for access by a remote caregiver.

In accordance with another aspect of the invention, a patient is allowed non-encumbered movement at home while daily-activity data of the patient is automatically, substantially continuously measured. A determination is made automatically from the daily-activity data of the patient whether the patient needs one or more of human investigation, human diagnosis, and/or human treatment. An alert to a remote caregiver is generated automatically only upon the determination from the daily-activity data of the patient that the patient needs the one or more of human investigation, human diagnosis, and/or human treatment.

In accordance with a further aspect of the invention, an article comprises one or more computer-readable signal-bearing media. The article includes means in the one or more media for making a determination from daily-activity data of a patient allowed freedom of movement at home whether the patient needs one or more of human investigation, human diagnosis, and/or human treatment. The article includes means in the one or more media for generating an indication of a change in condition of the patient for access by a remote caregiver upon the determination from the daily-activity data of the patient that the patient needs the one or more of human investigation, human diagnosis, and/or human treatment.

Various other features and advantages of the present invention will be made apparent from the following detailed description and the drawings.

### BRIEF DESCRIPTION OF THE DRAWINGS

The drawings illustrate a preferred embodiment presently contemplated for carrying out the invention.

In the drawings:

FIG. 1 is a representation of an implementation of an apparatus that comprises a device, a base station, a plurality of databases, an analyzer, and a display.

FIG. 2 is a representation of an exemplary logic flow for an implementation of the apparatus of FIG. 1.

FIG. 3 is a representation of a patient located in a home and illustrates an exemplary employment of the device of the apparatus of FIG. 1 and other devices.

### DETAILED DESCRIPTION OF THE PREFERRED EMBODIMENT

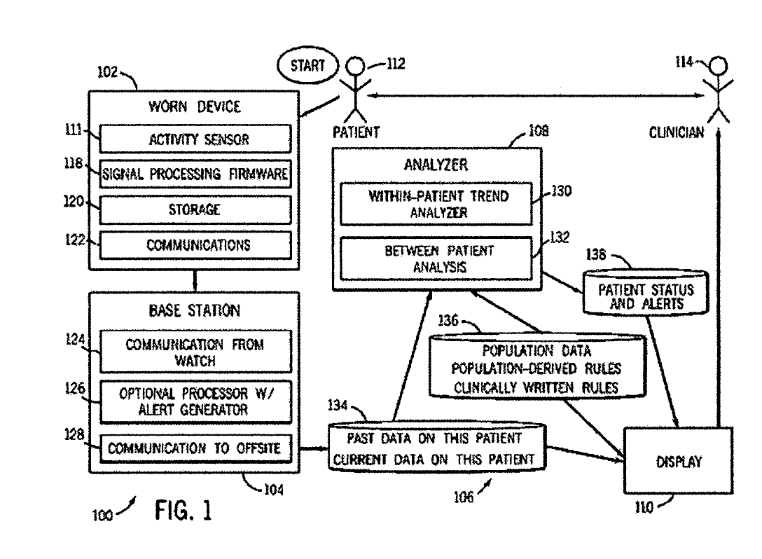

Turning to FIG. 1, an implementation of an apparatus 100 in an example comprises one or more of one or more devices 102, one or more base stations 104, one or more databases 106, one or more analyzers 108, and/or one or more displays 110. The apparatus 100 comprises a device 102, a base station 104, a plurality of databases 106, an analyzer 108, and a display 110. The device 102 comprises a user-wearable, non-encumbering device. The device 102 comprises an activity and/or a movement sensor 111 that substantially continuously measures daily-activity data of a patient 112. The movement sensor 111 comprises a user-wearable, non-encumbering, movement sensor. The analyzer 108 makes a determination from the daily-activity data of the patient 112 that a change in condition of the patient 112 has occurred. The analyzer 108 generates an indication of the change in condition of the patient 112 for access by a remote caregiver 114. The analyzer 108 generates an alert to the remote caregiver 114 only upon a determination from the daily-activity data of the patient 112 that the patient 112 needs medical review. The analyzer 108 generates the alert to the remote caregiver 114 only upon a determination from the daily-activity data of the patient 112 that a substantial change is taking place which may require medical review. Exemplary medical review occurs within two to three days of receipt of the indication of the change in condition. An exemplary need of the patient 112 is a need for non-emergency medical attention. The remote caregiver 114 is presented trends over days and/or weeks to verify treatment effectiveness or stabilization of condition of the patient 112.

The device 102 allows the patient 112 non-encumbered movement at home while automatically, substantially continuously measuring daily-activity data of the patient 112. The device 102 comprises the movement sensor 111 and one or more of signal processing firmware 118, storage 120, and/or transmitter 122. The device 102 in one example comprises the analyzer 108. The device 102 measures one or more of movement, acceleration, vibration, and/or change in orientation. Exemplary devices 102 comprise one or more of an accelerometer, actigraph, tilt switch, and/or motion detector. The device 102 comprises an accelerometer and/or an actigraph capable of measuring over twenty-four hour periods for a relatively long-term duration. The device 102 automatically, substantially continuously measures the daily-activity data of the patient 112. The device 102 performs substantially continuous measurements of the patient 112 as an indirect measure of health of the patient 112.

The device comprises a user-wearable, non-encumbering, in-home monitor that substantially continuously measures the daily-activity data of the patient 112. The device 102 comprises one or more of a convenient piece of jewelry, pendant, ornament, necklace, bracelet, timepiece, watch, item suspended from the patient 112, and/or item easily wearable by the patient 112. The device 102 comprises a strap for attachment to a wrist of the patient 112. The device 102 comprises a chain and/or other item for hanging the device 102 from a neck of the patient 112. The device 102 comprises one or more of a hook, clip, pin, an item for wearing the device 102 on a belt and/or clothing of the patient, and/or an item embedded within the clothing of the patient 112. The device 102 comprises one or more of a home-based monitor, watch, and/or pendant rather than a device pasted on the chest of a patient in a lab or hospital. The analyzer 108 makes a determination from the daily-activity data of the patient 112 that the change in condition of the patient 112 has occurred. The apparatus 100 comprises an automated system of the analyzer 108 providing the indication of the change in condition of the patient 112 for access by the remote caregiver 114. The apparatus 100 comprises an automated system of the analyzer 108 providing an alert to a clinician as the remote caregiver 114 of only one or more patients in need of one or more of human investigation, diagnosis, and/or treatment.

The device 102 comprises a collector that comprises the signal processing firmware 118 and/or the storage 120. The movement sensor 111 and the collector of the device 102 comprise a unitary construction and/or an integral formation. The collector of the device 102 receives the daily-activity data of the patient 112 from the movement sensor 111 with the signal processing firmware 118 and collects the daily-activity data of the patient 112 in the storage 120 on a substantially daily basis as daily-collection data for the patient 112. The collector of the device 102 provides the daily-activity data of the patient 112 to the transmitter 122. The collector of the device 102 stores the daily-activity data of the patient 112 in the storage 120 for later transmission by the transmitter 122 when the collector of the device 102 is out of range of the base station 104 that provides the daily-activity data of the patient 112 to the analyzer 108 approximately and/or substantially once per day.

The transmitter 122 comprises a communications resource. The transmitter 122 provides the daily-activity data of the patient 112 to the base station 104 from the collector of the device 102 approximately and/or substantially once per day. The transmitter 122 comprises a radio transmitter that transmits the daily-activity data of the patient 112 to the base station 104. The transmitter 122 comprises a transmitter that docks with the base station 104 to transmit the daily-activity data of the patient 112. The base station 104 involves simple zero or one-button operation to receive the daily-activity data of the patient 112 from the transmitter 122.

The base station 104 receives the daily-activity data of the patient 112 from the transmitter 122 of the device 102 and provides the daily-activity data of the patient 112 to the analyzer 108 approximately and/or substantially once per day. The base station 104 comprises one or more of a receiver 124, a processor 126, and/or a transmitter 128. The base station 104 in one example comprises the analyzer 108. The receiver 124 and/or the transmitter 128 comprise one or more communications resources. The databases 106 comprise one or more of a past and current patient data database 134, a population data and rules database 136, and/or a patient status and alert database 138. One or more of the past and current patient data database 134, the population data and rules database 136, and/or the patient status and alert database 138 may be combined in a single database 106.

The past and current patient data database 134 holds the activity data, past and present, for a patient 112 along with results of analysis. The population data and rules database 136 holds population models that assign meaning to activity values, shifts, and/or trends of the patient 112. These meanings could be derived empirically from studying other patients and/or outcomes. There could also be rules written based on clinical knowledge. The patient status and alert database 138 holds trends and alerts that potentially need to be reviewed by the remote caregivers 114.

The analyzer 108 is located on one or more of the device 102, the base station 104, and/or a network of the apparatus 100. The analyzer 108 occupies any physical location. The data of the patient 112 is located at any physical location. The data of the patient 112 is distributed or centralized. The links in the apparatus 100 employ any type of communication.

The analyzer 108 performs a baseline versus substantially continuous comparison of movement and/or activity data on the patient 112 from home as an indirect measure of health of the patient 112. The analyzer 108 is predictive of the health of the patient 112. The analyzer 108 employs daily-activity data of the patient 112 as a predictor whether the patient 112 is in need of medical intervention and/or attention. The analyzer 108 employs the daily-activity data of the patient 112 in making a probabilistic estimate of future events related to the health of the patient 112.

The analyzer 108 processes and simplifies the daily-activity data of the patient 112. The analyzer 108 automatically identifies changes in the activity data of the patient 112. The analyzer 108 looks for activity trends. The analyzer 108 automatically identifies changes in the activity data of a plurality of patients and picks out patients for possible attention so the remote caregiver 114 need not look at all the patients in a volume health care environment. The analyzer 108 classifies data as normal or abnormal to identify only patients who exhibit the biggest trends or shifts in activity for diagnosis by a health care professional as the remote caregiver 114.

The analyzer 108 comprises an individual patient analyzer 130 and/or a patient group analyzer 132. The analyzer 108 makes a determination from the daily-activity data of the patient 112 that the change in condition of the patient 112 has occurred. The analyzer 108 generates the indication of the change in condition of the patient 112 for access by the remote caregiver 114. The analyzer 108 generates the alert to the remote caregiver 114 only upon a determination from the daily-activity data of the patient 112 that the patient 112 needs medical review. The analyzer 108 makes a determination from the daily-collection data for the patient 112 that the change in condition of the patient 112 has occurred. The analyzer 108 generates the alert to the remote caregiver 114 only upon a determination from the daily-collection data for the patient 112 that the patient 112 needs medical review. The analyzer 108 automatically generates the alert to the remote caregiver 114 only upon an automated determination from the daily-activity data of the patient 112 that the patient 112 needs one or more of human investigation, human diagnosis, and/or human treatment. In a further example, the device 102, the base station 104, the database 106, the analyzer 108, and/or the display 110 serve to verify that a patient 112 is recovering or stable.

The analyzer 108 compares the daily-activity data of the patient 112 with previous-activity data of the patient 112 to make the determination that the change in condition of the patient 112 has occurred. The analyzer 108 compares the daily-activity data of the patient 112 with previous-activity data of the patient 112 to make the determination that the patient 112 needs medical review. The individual patient analyzer 130 considers one or more health trends of the patient 112 relative to the past health data on the patient 112. The analyzer 108 employs the daily-activity data of the patient 112 and previous-activity data of the patient 112 to look for one or more trends to make the determination that the change in condition of the patient 112 has occurred. The analyzer 108 employs the daily-activity data of the patient 112 and previous-activity data of the patient 112 to look for one or more trends to make the determination that the patient 112 needs medical review. The patient group analyzer 132 considers one or more health trends of the patient 112 relative to one or more approximately and/or substantially similar patients. The analyzer 108 employs the daily-activity data of the patient 112 and activity data of the one or more approximately and/or substantially similar patients to make the determination that the change in condition of the patient 112 has occurred. The analyzer 108 employs the daily-activity data of the patient 112 and activity data of the one or more approximately and/or substantially similar patients to make the determination that the patient 112 needs medical review.

The analyzer 108 generates the indication of the change in condition of the patient 112 for access by the remote caregiver 114. The analyzer 108 generates the alert to the remote caregiver 114 only upon a determination from the daily-activity data of the patient 112 that the patient 112 has a change in levels or patterns of activity or inactivity. The analyzer 108 makes an estimate whether medical review is needed based on an increased risk of an impending acute event for the patient 112. The analyzer 108 generates in the alert for the remote caregiver 114 a rank from the daily-activity data of the patient 112 of a need of the patient 112 for medical review. The analyzer 108 generates the alert to a remote clinician as the remote caregiver 114 only upon a determination from the daily-activity data of the patient 112 of a need for medical review due to an increased likelihood or an impending acute event for the patient 112.

The analyzer 108 makes a determination automatically from the daily-activity data of the patient 112 whether the patient 112 needs one or more of human investigation, human diagnosis, and/or human treatment. The analyzer 108 generates automatically an alert to the remote caregiver 114 only upon the determination from the daily-activity data of the patient 112 that the patient 112 needs the one or more of human investigation, human diagnosis, and/or human treatment. The analyzer 108 assigns to the patient 112 automatically from the daily-activity data of the patient 112 a ranked need of the patient 112 for the one or more of human investigation, human diagnosis, and/or human treatment in the alert to the remote caregiver 114. The analyzer 108 employs the display 110 to one or more of show, communicate, illustrate, depict, and/or represent to a health care professional automatically a ranked need of the patient 112 for the one or more of human investigation, human diagnosis, and/or human treatment in the alert relative to one or more other ranked needs of corresponding one or more other patients for one or more of human investigation, human diagnosis, and/or human treatment. The analyzer 108 assigns automatically a priority for one or more of human investigation, human diagnosis, and/or human treatment to one or more selected patients of the patient 112 and the one or more other patients based on the ranked need of the patient 112 and the one or more other ranked needs of the corresponding one or more other patients. The analyzer 108 alerts automatically a remote caregiver 114 of the priority for one or more of human investigation, human diagnosis, and/or human treatment of the one or more selected patients.

The analyzer 108 makes a determination from daily-activity data of a patient 112 allowed freedom of movement at home whether the patient 112 needs one or more of human investigation, human diagnosis, and/or human treatment. The analyzer 108 generates an alert to a remote caregiver 114 only upon the determination from the daily-activity data of the patient 112 that the patient 112 needs the one or more of human investigation, human diagnosis, and/or human treatment. The analyzer 108 assigns a priority for one or more of human investigation, human diagnosis, and/or human treatment to one or more selected patients of the patient 112 and one or more other patients based on a ranked need of the patient 112 and one or more other ranked needs of the one or more other patients. The analyzer 108 alerts a remote caregiver 114 of the priority for one or more of human investigation, human diagnosis, and/or human treatment of the one or more selected patients.

The remote caregiver 114 views the ranked results of the patient 112 from the analyzer 108 on the display 110. The analyzer 108 employs the daily-activity data of the patient 112 to generate the alert to the remote caregiver 114. The daily-activity data of the patient 112 sits in storage where the analyzer 108 employed the daily-activity data of the patient 112 while the alert goes to the remote caregiver 114. The entire dataset need not be transmitted past wherever the analysis sits. The analysis by the analyzer 108 allows clinicians as the remote caregivers 114 the efficiency of viewing only patients who have changed the most or look the worst compared to others. The efficiency saves time for the clinicians in avoidance of viewing other patients not identified as having as significant of needs as the selected patient 112. The remote caregiver 114 views on the display 110 the ranked results of the patient 112 from the analyzer 108 along with ranked results of one or more other patients from one or more other analyzers.

From the analysis by the analyzer 108, the remote caregiver 114 is triggered and/or makes a decision to one or more of investigate, diagnosis, and/or treat the patient 112 and/or one or more other patients. A clinician as the remote caregiver 114 employs one or more techniques to make inquiry of the patient 112. The remote caregiver 114 screens the patient 112 with a phone call. The remote caregiver 114 interviews the patient 112. The remote caregiver 114 performs question and answer and changes treatment for the patient 112 based on output from the analyzer 108 on the display 110. Based on the analysis of the patient 112 from the analyzer 108 and/or the result from inquiry of the patient 112 by the remote caregiver 114, the remote caregiver employs traditional and/or advanced medical techniques to diagnose and/or recommend changes to treatment of the patient 112 before onset of an acute event.

In-home activity monitoring can be used to detect changes in CHF condition, increasing quality of chronic disease management. If a patient 112 starts one or more of slowing down, sleeping in, not getting dressed, and/or tiring easily, the patient 112 and/or a person close to the patient 112 may or may not report the change and get care for the patient 112. The apparatus 100 automates the measurement of the patient 112, quantifies the results of the measurement, and use the analysis to alert a professional that something is going wrong with the patient 112. The apparatus can verify improvement after a treatment of the patient 112 and show that the improvement is maintained.

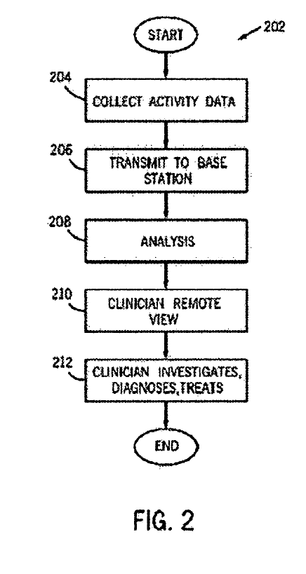

An illustrative description of an exemplary operation of an implementation of the apparatus 100 is presented, for explanatory purposes. Turning to FIG. 2, in exemplary logic flow 202 at STEP 204, the collector of the device 102 collects activity data of the patient 112. At STEP 206, the transmitter 122 of the device 102 transmits the daily-activity data of the patient 112 to the base station 104. At STEP 208, the analyzer 108 employs the individual patient analyzer 130 and/or the patient group analyzer 132 to consider one or more activity trends of the patient 112. At STEP 210, the remote caregiver 114 views the ranked results of the patient 112 from the analyzer 108 on the display 110. The remote caregiver 114 views on the display 110 the ranked results of the patient 112 from the analyzer 108 along with ranked results of one or more other patients from one or more other analyzers. At STEP 212, the remote caregiver 114 is triggered and/or makes a decision to one or more of investigate, diagnosis, and/or treat the patient 112 and/or one or more other patients.

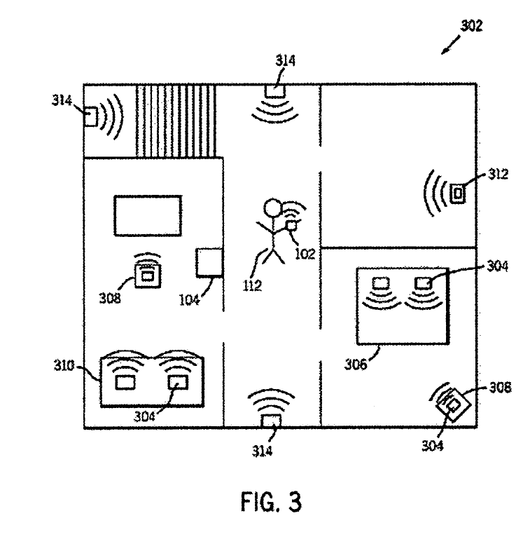

Turning to FIG. 3, the patient 112 is located in a home 302. The device 102 is employable with other worn devices and/or other devices placed around the home 302 of the patient 112. The device 102 helps identify the patient 112 when the device 102 is employed with other devices for measuring activity of the patient 112. Pressure pads and/or sensors 304 are located in a bed 306, chairs 308, a sofa 310, and/or a scale 312 of the patient 112. Motions sensors 314 are located in the rooms, hallways, doorways, and/or stairways of the home 302. The device 102, pressure sensors 304, and motions sensors 314 communicate with the base station 104 that provides the daily-activity data of the patient 112 to the analyzer 108 (FIG. 1) approximately and/or substantially once per day. The analyzer 108 is located on one or more of the device 102, the base station 104, and/or a network of the apparatus 100. The analyzer 108 employs daily-activity data of the patient 112 as a predictor whether the patient 112 is in need of medical intervention and/or attention. The analyzer 108 makes a determination from the daily-collection data for the patient 112 that the change in condition of the patient 112 has occurred. The analyzer 108 generates the alert to the remote caregiver 114 only upon a determination from the daily-collection data for the patient 112 that the patient 112 needs medical review. The device 102 is employable with additional medical measurements, for example, blood pressure and weight of the patient 112.

An exemplary device 102 monitors and/or provides indirect measures of sleep quality and/or caloric expenditure and/or provides authentication when correlated with other sensors. Exemplary CHF symptoms correlated with measures by the device 102 comprise chest pain, coughing, difficulty breathing, shortness of breath, fatigue, and/or swollen legs. Exemplary pressure sensors 304 monitor sleep quality, sleep location, and/or information on sedentary activities. Exemplary CHF symptoms correlated with measures by the pressure sensors 304 comprise inability to sleep in one position and/or fluid retention. Exemplary motion sensors 314 monitor walking speed and/or bathroom usage. The motion sensors 314 are positioned at both ends of a passage where lingering is at a minimum, for example, at opposite ends of a hallway. Exemplary CHF symptoms correlated with measures by the motion sensors 314 comprise shortness of breath, fatigue, and/or swollen legs.

Exemplary intra-patient processing by the device 102 and/or the analyzer 108 comprises a determination of a baseline for the patient 112 at hospital release, tracking of improvement in health of the patient 112, and alerting the remote caregiver 114 when the improvement in health of the patient 112 is not sustained and/or activity of the patient 112 drops back towards immediate post-hospital activity data of the patient 112. Exemplary inter-patient processing sub-groups patients with one or more of approximately and/or substantially similar diagnoses, activity levels, activity patterns, and/or circadian rhythms. Further exemplary inter-patient processing normalizes data on the patients to points in disease progression and/or days since and/or until next treatment for acute condition.

The patient 112 is allowed non-encumbered movement at the home 302 while automatically, substantially continuously measuring daily-activity data of the patient 112. A determination is made automatically from the daily-activity data of the patient 112 whether the patient 112 needs one or more of human investigation, human diagnosis, and/or human treatment. An alert to the remote caregiver 114 is generated automatically only upon the determination from the daily-activity data of the patient 112 that the patient 112 needs the one or more of human investigation, human diagnosis, and/or human treatment. The patient 112 is assigned automatically from the daily-activity data of the patient 112 a ranked need of the patient 112 for the one or more of human investigation, human diagnosis, and/or human treatment in the alert to the remote caregiver 114. In the alert to a health care professional as the remote caregiver 114, a ranked need of the patient 112 for one or more of human investigation, human diagnosis, and/or human treatment relative to one or more other ranked needs of corresponding one or more other patients for one or more of human investigation, human diagnosis, and/or human treatment is automatically displayed on the display 110. A priority is assigning automatically for one or more of human investigation, human diagnosis, and/or human treatment to one or more selected patients of the patient 112 and the one or more other patients based on the ranked need of the patient 112 and the one or more other ranked needs of the corresponding one or more other patients. A medical doctor as the remote caregiver 114 is alerted automatically of the priority for the one or more of human investigation, human diagnosis, and/or human treatment of the one or more selected patients.

The device 102 is employable with additional medical measurements, for example, blood pressure and weight of the patient 112.

An implementation of the apparatus 100 in an example comprises a plurality of components such as one or more of electronic components, hardware components, and/or computer software components. A number of such components can be combined or divided in an implementation of the apparatus 100. An exemplary component of an implementation of the apparatus 100 employs and/or comprises a set and/or series of computer instructions written in or implemented with any of a number of programming languages, as will be appreciated by those skilled in the art. An implementation of the apparatus 100 in an example comprises any (e.g., horizontal, oblique, or vertical) orientation, with the description and figures herein illustrating an exemplary orientation of an implementation of the apparatus 100, for explanatory purposes.

An implementation of the apparatus 100 in an example employs one or more computer-readable signal-bearing media. A computer-readable signal-bearing medium in an example stores software, firmware and/or assembly language for performing one or more portions of one or more implementations. An example of a computer-readable signal bearing medium for an implementation of the apparatus 100 comprises the recordable data storage medium as the storage 120 of the device 102 and/or a recordable data storage medium of the analyzer 108. A computer-readable signal-bearing medium for an implementation of the apparatus 100 in an example comprises one or more of a magnetic, electrical, optical, biological, and/or atomic data storage medium. For example, an implementation of the computer-readable signal-bearing medium comprises floppy disks, magnetic tapes, CD-ROMs, DVD-ROMs, hard disk drives, and/or electronic memory. In another example, an implementation of the computer-readable signal-bearing medium comprises a modulated carrier signal transmitted over a network comprising or coupled with an implementation of the apparatus 100, for instance, one or more of a telephone network, a local area network (“LAN”), a wide area network (“WAN”), the Internet, and/or a wireless network.

The steps or operations described herein are examples. There may be variations to these steps or operations without departing from the spirit of the invention. For example, the steps may be performed in a differing order, or steps may be added, deleted, or modified.

The present invention has been described in terms of a preferred embodiment, and it is recognized that equivalents, alternatives, and modifications, aside from those expressly stated, are possible and within the scope of the appending claims.

What is claimed is:

1. An apparatus, comprising:
a user-wearable, non-encumbering, movement sensor that substantially continuously measures daily-activity data of a patient; and
an analyzer that makes a determination from the daily-activity data of the patient that a change in condition of the patient has occurred, wherein the analyzer generates an indication of the change in condition of the patient for access by a remote caregiver.

2. The apparatus of claim 1, wherein the analyzer compares the daily-activity data of the patient with previous-activity data of the patient to make the determination that the change in condition of the patient has occurred.

3. The apparatus of claim 1, wherein the analyzer employs the daily-activity data of the patient and previous-activity data of the patient to look for one or more trends to make the determination that the change in condition of the patient has occurred.

4. The apparatus of claim 1, wherein the analyzer employs the daily-activity data of the patient and activity data of one or more approximately and/or substantially similar patients to make the determination that the change in condition of the patient has occurred.

5. The apparatus of claim 1, wherein the user-wearable, non-encumbering, movement sensor automatically, substantially continuously measures the daily-activity data of the patient;
wherein the analyzer automatically generates the indication of the change in condition of the patient for access by the remote caregiver only upon an automated determination from the daily-activity data of the patient that the patient needs one or more of human investigation, human diagnosis, and/or human treatment.

6. The apparatus of claim 1, wherein the analyzer generates the indication of the change in condition of the patient for access by the remote caregiver upon a determination from the daily-activity data of the patient that the patient has a change in chronic heart failure (CHF) condition.

7. The apparatus of claim 1, wherein the analyzer makes an estimate whether one or more of hospitalization is needed, an acute event is impending, and/or death is impending for the patient.

8. The apparatus of claim 1, wherein the analyzer generates in the indication of the change in condition of the patient for access by the remote caregiver a rank from the daily-activity data of the patient of a need of the patient for medical review.

9. The apparatus of claim 1, wherein the analyzer generates the indication of the change in condition of the patient for access by the remote caregiver upon a determination from the daily-activity data of the patient of likelihood that one or more of hospitalization is needed, an acute event is impending, and/or death is impending for the patient.

10. The apparatus of claim 1, wherein the user-wearable, non-encumbering, movement sensor comprises a user-wearable, non-encumbering, in-home monitor that substantially continuously measures the daily-activity data of the patient.

11. The apparatus of claim 1, further comprising:
a collector that receives the daily-activity data of the patient from the user-wearable, non-encumbering, movement sensor and collects the daily-activity data of the patient on a substantially daily basis as daily-collection data for the patient;
wherein the analyzer generates the indication of the change in condition of the patient for access by the remote caregiver upon a determination from the daily-collection data for the patient that the change in condition of the patient has occurred.

12. The apparatus of claim 11, wherein the user-wearable, non-encumbering, movement sensor and the collector comprise a unitary construction and/or an integral formation.

13. The apparatus of claim 11, wherein the collector stores the daily-activity data of the patient for later transmission when the collector is out of range of a base station that provides the daily-activity data of the patient to the analyzer approximately and/or substantially once per day.

14. The apparatus of claim 1, further comprising:
a transmitter that comprises one of:
a radio transmitter located on a user-wearable, non-encumbering device that comprises the user-wearable, non-encumbering, movement sensor; or
a base station that provides the daily-activity data of the patient to the analyzer approximately and/or substantially once per day.

15. A method, comprising the steps of:
allowing a patient non-encumbered movement at home while automatically, substantially continuously measuring daily-activity data of the patient;
making a determination automatically from the daily-activity data of the patient whether the patient needs one or more of human investigation, human diagnosis, and/or human treatment; and
generating automatically an alert to a remote caregiver only upon the determination from the daily-activity data of the patient that the patient needs the one or more of human investigation, human diagnosis, and/or human treatment.

16. The method of claim 15, wherein the step of generating automatically the alert to the remote caregiver comprises the step of:
assigning to the patient automatically from the daily-activity data of the patient a ranked need of the patient for the one or more of human investigation, human diagnosis, and/or human treatment in the alert to the remote caregiver.

17. The method of claim 15, further comprising the step of:
displaying to a health care professional automatically a ranked need of the patient for the one or more of human investigation, human diagnosis, and/or human treatment in the alert relative to one or more other ranked needs of corresponding one or more other patients for one or more of human investigation, human diagnosis, and/or human treatment.

18. The method of claim 17, wherein the step of generating automatically the alert to the remote caregiver comprises the steps of:
assigning automatically a priority for one or more of human investigation, human diagnosis, and/or human treatment to one or more selected patients of the patient and the one or more other patients based on the ranked need of the patient and the one or more other ranked needs of the corresponding one or more other patients; and
alerting automatically a medical doctor of the priority for the one or more of human investigation, human diagnosis, and/or human treatment of the one or more selected patients.

19. An article, comprising:
one or more computer-readable signal-bearing media; and
means in the one or more media for making a determination from daily-activity data of a patient allowed freedom of movement at home whether the patient needs one or more of human investigation, human diagnosis, and/or human treatment; and
means in the one or more media for generating an indication of a change in condition of the patient for access by a remote caregiver upon the determination from the daily-activity data of the patient that the patient needs the one or more of human investigation, human diagnosis, and/or human treatment.

20. The article of claim 19, wherein the means in the one or more media for generating the indication of the change in condition of the patient for access by the remote caregiver comprises:
means in the one or more media for assigning a priority for one or more of human investigation, human diagnosis, and/or human treatment to one or more selected patients of the patient and one or more other patients based on a ranked need of the patient and one or more other ranked needs of the one or more other patients; and
means in the one or more media for alerting a medical doctor of the priority for the one or more of human investigation, human diagnosis, and/or human treatment of the one or more selected patients.
